(19) United States
(12) Patent Application Publication
(10) Pub. No.: US 2021/0057045 A1
(43) Pub. Date: Feb. 25, 2021

(54) Determining the Clinical Significance of Variant Sequences

(71) Applicant: Good Start Genetics, Inc., Cambridge, MA (US)

(72) Inventors: Caleb J. KENNEDY, Arlington, MA (US); Mark UMBARGER, Brookline, MA (US); Gregory PORRECA, Cambridge, MA (US)

(21) Appl. No.: 16/997,517

(22) Filed: Aug. 19, 2020

Related U.S. Application Data
(63) Continuation of application No. 13/488,142, filed on Jun. 4, 2012, now abandoned.

(51) Int. Cl. G16B 30/00

(52) U.S. Cl. CPC G16B 30/00; G16B 20/00

(57) ABSTRACT

The present invention generally relates to determining the clinical significance of a variant nucleic acid sequence. The invention can involve sequencing a nucleic acid to generate at least one sequence read, identifying a variant sequence within the sequence read, determining the equivalent insertion/deletion region (EIR) of the variant sequence, identifying a functional region including at least a portion of the EIR, and associating the EIR with the identified functional region, thereby to determine the clinical significance of the variant.

### RELATED APPLICATIONS

This application is a continuation of U.S. patent application Ser. No. 13/488,142, filed Jun. 4, 2012, which is herein incorporated by reference in its entirety.

### FIELD OF THE INVENTION

The present invention generally relates to determining the clinical significance of certain mutations in genetic sequences.

### BACKGROUND

Methods of sequencing or identifying significant fractions of the human genome and genetic variations within those fractions are becoming commonplace. The methods are used, for example, in genetic counseling, where a person's genetic make-up is studied to determine potential clinical impact. Sequencing provides an important step towards reaching that determination.

Next-generation sequencing (NGS) technologies include instruments capable of sequencing more than 1014 kilobase-pairs (kbp) of DNA per instrument run. Sequencing typically produces a large number of independent reads, each representing anywhere between 10 to 1000 bases of the nucleic acid. Nucleic acids are generally sequenced redundantly for confidence, which replicates per unit area being referred to as the coverage (i.e., “10× coverage” or “100× coverage”). Thus, a multi-gene genetic screening can produce millions of reads.

Insertion and deletion variants present a particular challenge for high-throughput screening technologies. These variants can originate, for example, from sequencing artifacts in the form of false positive mutations. Aligning reads with coordinate-altering variants requires the use of penalized gaps in either the query or reference sequence to maintain global coordinate order. Gaps are often inserted at the ends of reads to artificially maintain optimality, leading to false positive insertion, deletion, and substitution variants. Realignment improves the sensitivity of insertions/deletions and specificity of substitutions, however, these techniques often use Smith-Waterman alignment algorithms without gaps. Without penalizing gaps, false positive insertions and deletions often result.

Conversely, variants can also constitute true mutations that arise from genetic events. For example, small insertions and deletions (<100 bp) commonly occur within tandem repeats where polymerase slippage or intra-chromosomal recombination leads to nucleotide expansion or contraction. Relative to the original (or reference genome), the consequences of these processes appear as insertions or deletions, respectively.

The inability to distinguish between those variants that are merely sequencing artifacts and those that are true mutations hinders efforts to acquire a better understanding of sequencing data. In addition, for those variant sequences that are indeed true mutations, their clinical significance is not readily known if the variant is novel, i.e., not previously characterized. This is particularly the case for variant sequences that are not associated with the most common disease-causing mutations prevalent in specific populations.

### SUMMARY

The present invention generally relates to determining the clinical significance of a variant sequence. It has been found that by correlating the positional sequence information associated with a variant sequence to the functional context associated with the sequence, the clinical impact of the variant can be determined. Accordingly, methods of the invention permit the determination of a clinical effect of a variant sequence, even when the genomic location of the variant is ambiguous. The ability to interpret variants observed in sequence data and to determine their clinical relevance allows rapid, cost-effective analysis of large datasets encountered in next-generation sequencing. Methods of the invention encompass sequencing a nucleic acid to generate at least one sequence read, identifying a variant sequence within the sequence read based on mapping the read to a reference sequence, determining the equivalent insertion/deletion region of the variant sequence, identifying a functional region that includes at least a portion of the equivalent insertion/deletion region, and associating the equivalent insertion/deletion region with the identified functional region, thereby to determine the clinical significance of the variant.

Generally, nucleic acid obtained from a subject is sequenced to produce a plurality of reads. Sequencing the nucleic acid may be by any method known in the art. For example, sequencing may involve pyrosequencing, sequencing-by-synthesis, reversible dye-terminators, or sequencing by hybridization.

Once the sequence reads are obtained, variant sequences within the sequence reads are identified. Generally, this is performed by aligning or mapping the sequence read to another sequence. In certain aspects, this other sequence is a reference sequence. Various algorithms can be used to map the sequence reads to a reference genome. Additional algorithms can be used to detect variants in the sequence reads based on the mapping results.

As mentioned above, the methods of the invention exploit the genomic coordinate space of the variant sequence in addition to the protein coding space associated with the variant sequence in order to determine clinical significance. The genomic coordinate space of the variant is obtained by determining the equivalent insertion/deletion region or EIR of the variant. The EIR represents all the equivalent positions for a particular insertion or deletion in a given sequence where that particular insertion or deletion cannot be unambiguously defined by a single position. Methods of determining the EIR of a variant sequence are known in the art, and typically encompass the use of various algorithms to perform the determinations.

Determination of the variant EIR provides additional information that is useful for understanding the nature of the variant. Methods of the invention include using the EIR to determine whether the true mutation associated with genetic processes or merely a false positive generated by sequencing artifacts. It has been found, for example, that an EIR with a length greater than one base pair is more likely to be associated with a true mutation. An EIR may be one base pair if the corresponding variant is simply a substitution and not an insertion/deletion or if the insertion or deletion occurs outside a tandem repetitive region, which by nature will have an EIR greater than one. Tandem repeats are known to be relatively unstable, with high rates of mutation. Accordingly, insertions or deletions that occur in these regions are more likely to be true mutations.

Whereas the positional information associated with the variant is derived from determining the EIR, the functional context or region associated with the variant is determined by annotating the variant EIR with all the appropriate functional information. Functional information represents the relevant protein coding region. In certain aspects, the functional annotation regions are described using known gene structures, for example, genes, exons, introns, splice sites, codons, regulatory elements, non-coding regions and the like. Again, annotating the variant with the appropriate functional region can be performed using the proper algorithms.

The positional information is then associated with the functional context to determine the clinical impact of the variant sequence. In certain aspects, association involves determining whether the EIR extends beyond the functional region. If the variant EIR extends beyond the functional region, it can be pushed out of the functional region. Accordingly, the variant has no functional effect (and corresponding clinical impact) because it could lie outside the functional region. If the variant cannot be pushed out of the functional region, its clinical significance is then assessed.

Methods of the invention are particularly helpful when the variant sequences are novel. One could, for example, begin the determination of a variant's clinical impact by consulting a relevant database to characterize the variant. If the variant is not found in the database, i.e., it is a novel variant, one can still proceed with the methods outlined above to determine the clinical impact of the variant. The analysis would not have to come to an end simply because the variant has not been previously characterized.

Methods of the invention are also particularly helpful when the variant sequence is associated with a tandem repeat. Due to the nature of a tandem repeat, insertions or deletions occurring in the region may be positionally ambiguous. Methods of the invention account for this inherent ambiguity and nonetheless, can still provide meaningful information regarding the clinical impact of the insertion or deletion.

Other aspects of the invention will become apparent upon review of the following disclosure.

### BRIEF DESCRIPTION OF THE DRAWINGS

FIG. 1 depicts the alignment of a sequence containing a homopolymeric repeat.

FIG. 2 is a process chart depicting an algorithm for determining the clinical significance of a variant sequence, according to certain embodiments.

FIG. 3 illustrates a system for performing methods of the invention.

### DETAILED DESCRIPTION

The invention generally relates to determining the clinical significance of a variant sequence. The invention can involve sequencing a nucleic acid to generate at least one sequence read and identifying a variant sequence within the sequence read to a reference sequence. The equivalent insertion/deletion region (EIR) of the variant sequence then can be determined. And then a functional region comprising at least a portion of the equivalent insertion/deletion region can be identified. The clinical significance of the variant sequence is determined by associating the EIR with the identified functional region.

Nucleic acid in a sample can be any nucleic acid, including for example, genomic DNA in a tissue sample, cDNA amplified from a particular target in a laboratory sample, or mixed DNA from multiple organisms. In some embodiments, the sample includes homozygous DNA from a haploid or diploid organism. For example, a sample can include genomic DNA from a patient who is homozygous for a rare recessive allele. In other embodiments, the sample includes heterozygous genetic material from a diploid or polyploidy organism with a somatic mutation such that two related nucleic acids are present in allele frequencies other than 50 or 100%, i.e., 20%, 5%, 1%, 0.1%, or any other allele frequency.

In one embodiment, nucleic acid template molecules (e.g., DNA or RNA) are isolated from a biological sample containing a variety of other components, such as proteins, lipids, and non-template nucleic acids. Nucleic acid template molecules can be obtained from any cellular material, obtained from animal, plant, bacterium, fungus, or any other cellular organism. Biological samples for use in the present invention also include viral particles or preparations. Nucleic acid template molecules can be obtained directly from an organism or from a biological sample obtained from an organism, e.g., from blood, urine, cerebrospinal fluid, seminal fluid, saliva, sputum, stool, and tissue. Any tissue or body fluid specimen may be used as a source for nucleic acid to use in the invention. Nucleic acid template molecules can also be isolated from cultured cells, such as a primary cell culture or cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA from the freezer.

Generally, nucleic acid can be extracted from a biological sample by a variety of techniques such as those described by Maniatis, et al., Molecular Cloning: A Laboratory Manual, 1982, Cold Spring Harbor, N.Y., pp. 280-281; Sambrook and Russell, Molecular Cloning: A Laboratory Manual 3Ed, Cold Spring Harbor Laboratory Press, 2001, Cold Spring Harbor, NY; or as described in U.S. Pub. 2002/01900663.

Nucleic acid obtained from biological samples may be fragmented to produce suitable fragments for analysis. Template nucleic acids may be fragmented or sheared to a desired length, using a variety of mechanical, chemical, and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid is fragmented by sonication. In another embodiment, nucleic acid is fragmented by a hydroshear instrument. Generally, individual nucleic acid template molecules can be from about 2 kb bases to about 40 kb. In a particular embodiment, nucleic acids are about 6 kb-10 kb fragments. Nucleic acid molecules may be single-stranded, double-stranded, or double stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In one embodiment, the concentration of the detergent is between 0.1% to about 2%. The detergent, particularly one that is mild and nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol. C12E8 (octaethylene glycol n-dodecyl monoether), hexaethyleneglycol mono-n-tetradecyl ether (CI4EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl) dimethyl-ammonio]-1-propanesulfonate. It is contemplated also that urea may be added with or without another detergent or surfactant.

Lysis or homogenization solutions may further contain other agents, such as reducing agents. Examples of such reducing agents include dithiothretol (DTT), β-mercaptoethanol, DTE, GSH, cysteine, cystemine, tricarboxyethyl phosphine (TCEP), or salts of sulfurous acid.

In various embodiments, the nucleic acid is amplified, for example, from the sample or after isolation from the sample. Amplification refers to production of additional copies of a nucleic acid sequence and is generally conducted using polymerase chain reaction (PCR) or other technologies well-known in the art (e.g., Dieffenbach and Dveksler, PCR Primer, a Laboratory Manual, 1995, Cold Spring Harbor Press, Plainview, N.Y.). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany, F. Genome research, 1:5-16 (1991); Barany, F., PNAS, 88:189-193 (1991); U.S. Pat. Nos. 5,869,252; and 6,100,099), strand displacement amplification and restriction fragment length polymorphism, transcription based amplification system, rolling circle amplification, and hyper-branched rolling circle amplification. Further examples of amplification techniques that can be used include, without limitation, quantitative PCR, quantitative fluorescent PCR (QF-PCR), multiplex fluorescent PCR (MF-PCR), real time PCR (RTPCR), single cell PCR, restriction fragment length polymorphism (PCR-RFLP). RT-PCR-RFLP, hot start PCR, in situ polonony PCR, in situ rolling circle amplification (RCA), bridge PCR, picotiter PCR, and emulsion PCR. Other suitable amplification methods include transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938. In certain embodiments, the amplification reaction is the polymerase chain reaction. Polymerase chain reaction refers to methods by K. B. Mullis (U.S. Pat. Nos. 4,683,195 and 4,683,202, hereby incorporated by reference) for increasing concentration of a segment of a target sequence in a mixture of genomic DNA without cloning or purification.

Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software from Arrayit Corporation (Sunnyvale, Calif.), Oligonucleotide Probe Sequence Design Software for Genetic Analysis from Olympus Optical Co., Ltd. (Tokyo, Japan), NetPrimer, and DNAsis Max v3.0 from Hitachi Solutions America, Ltd. (South San Francisco. Calif.). The TM (melting or annealing temperature) of each primer is calculated using software programs such as OligoAnalyzer 3.1 available on the web site of Integrated DNA Technologies. Inc. (Coralville, Iowa).

With PCR, it is possible to amplify a single copy of a specific target sequence in genomic DNA to a level that can be detected by several different methodologies (e.g., staining; hybridization with a labeled probe; incorporation of biotinylated primers followed by avidin-enzyme conjugate detection; or incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

Amplification adapters may be attached to the fragmented nucleic acid. Adapters may be commercially obtained, such as from Integrated DNA Technologies (Coralville, Iowa). In certain embodiments, the adapter sequences are attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase, available commercially from New England Biolabs (Ipswich, Mass.). Methods for using ligases are well known in the art. The polymerase may be any enzyme capable of adding nucleotides to the 3′ and the 5′ terminus of template nucleic acid molecules.

The ligation may be blunt ended or utilize complementary overhanging ends. In certain embodiments, the ends of the fragments may be repaired, trimmed (e.g. using an exonuclease), or filled (e.g., using a polymerase and dNTPs) following fragmentation to form blunt ends. In some embodiments, end repair is performed to generate blunt end 5′ phosphorylated nucleic acid ends using commercial kits, such as those available from Epicentre Biotechnologies (Madison, Wis.). Upon generating blunt ends, the ends may be treated with a polymerase and dATP to form a template independent addition to the 3′-end and the 5′-end of the fragments, thus producing a single A overhanging. This single A is used to guide ligation of fragments with a single T overhanging from the 5′-end in a method referred to as T-A cloning.

Alternatively, because the possible combination of overhangs left by the restriction enzymes are known after a restriction digestion, the ends may be left as-is, i.e., ragged ends. In certain embodiments double stranded oligonucleotides with complementary overhanging ends are used.

In certain embodiments, a single bar code is attached to each fragment. In other embodiments, a plurality of bar codes, e.g., two bar codes, are attached to each fragment.

A bar code sequence generally includes certain features that make the sequence useful in sequencing reactions. For example the bar code sequences are designed to have minimal or no homopolymer regions, i.e., 2 or more of the same base in a row such as AA or CCC, within the bar code sequence. The bar code sequences are also designed so that they are at least one edit distance away from the base addition order when performing base-by-base sequencing, ensuring that the first and last base do not match the expected bases of the sequence.

The bar code sequences are designed such that each sequence is correlated to a particular portion of nucleic acid, allowing sequence reads to be correlated back to the portion from which they came. Methods of designing sets of bar code sequences are shown for example in U.S. Pat. No. 6,235,475, the contents of which are incorporated by reference herein in their entirety. In certain embodiments, the bar code sequences range from about 5 nucleotides to about 15 nucleotides. In a particular embodiment, the bar code sequences range from about 4 nucleotides to about 7 nucleotides. Since the bar code sequence is sequenced along with the template nucleic acid, the oligonucleotide length should be of minimal length so as to permit the longest read from the template nucleic acid attached. Generally, the bar code sequences are spaced from the template nucleic acid molecule by at least one base (minimizes homopolymeric combinations).

Embodiments of the invention involve attaching the bar code sequences to the template nucleic acids. In certain embodiments, the bar code sequences are attached to the template nucleic acid molecule with an enzyme. The enzyme may be a ligase or a polymerase, as discussed above. Attaching bar code sequences to nucleic acid templates is shown in U.S. Pub. 2008/0081330 and U.S. Pub. 2011/0301042, the contents of which are incorporated by reference herein in its entirety. Methods for designing sets of bar code sequences and other methods for attaching bar code sequences are shown in U.S. Pat. Nos. 6,138,077; 6,352,828; 5,636,400; 6,172,214; 6,235,475; 7,393,665; 7,544.473; 5,846,719; 5,695,934; 5.604.097; 6,150,516; RE39,793; 7,537,897; 6172,218; and 5,863,722, the content of each of which is incorporated by reference herein in its entirety.

After any of the aforementioned processing steps (e.g., obtaining, isolating, fragmenting, or amplification), nucleic acid can be sequenced to generate a plurality of sequence reads, according to certain embodiments of the invention.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing. Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Sequencing of separated molecules has more recently been demonstrated by sequential or single extension reactions using polymerases or ligases as well as by single or sequential differential hybridizations with libraries of probes.

A sequencing technique that can be used in the methods of the provided invention includes, for example, 454 sequencing (454 Life Sciences, a Roche company, Branford, Conn.) (Margulies. M et al., Nature, 437:376-380 (2005); U.S. Pat. Nos. 5,583,024; 5,674,713; and 5,700,673). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology by Applied Biosystems from Life Technologies Corporation (Carlsbad, Calif.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing, described, for example, in U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H⁺), which signal is detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, the contents of which are herein incorporated by reference in their entirety.

Another example of a sequencing technology that can be used in the methods of the provided invention includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni, G. V., and Meller, A., Clin Chem 53: 1996-2001 (2007)). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in U.S. Pub. 2009/0026082). In one example of the technique. DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M., PNAS, 53:564-71 (1965)). In one example of the technique, individual DNA molecules are labeled using metallic labels that are distinguishable using an electron microscope. These molecules are then stretched on a flat surface and imaged using an electron microscope to measure sequences.

Sequencing according to embodiments of the invention generates a plurality of reads. Reads according to the invention generally include sequences of nucleotide data less than about 150 bases in length, or less than about 90 bases in length. In certain embodiments, reads are between about 80 and about 90 bases, e.g., about 85 bases in length. In some embodiments, methods of the invention are applied to very short reads, i.e., less than about 50 or about 30 bases in length. Further methods for processing of sequence reads, including the assembly of sequence reads into contigs, is described in detail in U.S. patent application Ser. No. 13/439,508, incorporated herein by reference. A contig, generally, refers to the relationship between or among a plurality of segments of nucleic acid sequences, e.g., reads. Where sequence reads overlap, a contig can be represented as a layered image of overlapping reads.

Certain embodiments of the invention provide for the assembly of sequence reads. In assembly by alignment, for example, the reads are aligned to each other or to a reference. By aligning each read, in turn to a reference genome, all of the reads are positioned in relationship to each other to create the assembly. In addition, aligning or mapping the sequence read to a reference sequence can also be used to identify variant sequences within the sequence read.

Computer programs for assembling reads are known in the art. Such assembly programs can run on a single general-purpose computer, on a cluster or network of computers, or on specialized computing devices dedicated to sequence analysis.

Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren, R., et al., Bioinformatics, 23:500-501 (2007)). SSAKE cycles through a table of reads and searches a prefix tree for the longest possible overlap between any two sequences. SSAKE clusters reads into contigs.

Another read assembly program is Forge Genome Assembler, written by Darren Platt and Dirk Evers and available through the SourceForge web site maintained by Geeknet (Fairfax, Va.) (see, e.g., DiGuistini, S., et al., Genome Biology, 10:R94 (2009)). Forge distributes its computational and memory consumption to multiple nodes, if available, and has therefore the potential to assemble large sets of reads. Forge was written in C++ using the parallel MPI library. Forge can handle mixtures of reads, e.g., Sanger, 454, and Illumina reads.

Assembly through multiple sequence alignment can be performed, for example, by the program Clustal Omega, (Sievers F., et al., Mol Syst Biol 7 (2011)), ClustalW, or ClustalX (Larkin M. A., et al., Bioinformatics, 23, 2947-2948 (2007)) available from University College Dublin (Dublin, Ireland).

Another exemplary read assembly program known in the art is Velvet, available through the web site of the European Bioinformatics Institute (Hinxton, UK) (Zerbino D. R. et al., Genome Research 18(5):821-829 (2008)). Velvet implements an approach based on de Bruijn graphs, uses information from read pairs, and implements various error correction steps.

Read assembly can be performed with the programs from the package SOAP, available through the website of Beijing Genomics Institute (Beijing, Conn.) or BGI Americas Corporation (Cambridge, Mass.). For example, the SOAPdenovo program implements a de Bruijn graph approach. SOAP3/GPU aligns short reads to a reference sequence.

Another read assembly program is ABySS, from Canada's Michael Smith Genome Sciences Centre (Vancouver, B.C., CA) (Simpson, J. T., et al., Genome Res., 19(6):1117-23 (2009)). ABySS uses the de Bruijn graph approach and runs in a parallel environment.

Read assembly can also be done by Roche's GS De Novo Assembler, known as gsAssembler or Newbler (NEW assemBLER), which is designed to assemble reads from the Roche 454 sequencer (described, e.g., in Kumar, S. et al., Genomics 11:571 (2010) and Margulies, et al., Nature 437:376-380 (2005)). Newbler accepts 454 Flx Standard reads and 454 Titanium reads as well as single and paired-end reads and optionally Sanger reads. Newbler is run on Linux, in either 32 bit or 64 bit versions. Newbler can be accessed via a command-line or a Java-based GUI interface.

Cortex, created by Mario Caccamo and Zamin Iqbal at the University of Oxford, is a software framework for genome analysis, including read assembly. Cortex includes cortex_con for consensus genome assembly, used as described in Spanu, P. D., et al., Science 330(6010):1543-46 (2010). Cortex includes cortex_var for variation and population assembly, described in Iqbal, et al., De novo assembly and genotyping of variants using colored de Bruijn graphs, Nature Genetics (in press), and used as described in Mills, R. E., et al., Nature 470:59-65 (2010). Cortex is available through the creators' web site and from the SourceForge web site maintained by Geeknet (Fairfax, Va.).

Other read assembly programs include RTG Investigator from Real Time Genomics, Inc. (San Francisco, Calif.); iAssembler (Zheng, et al., BMC Bioinformatics 12:453 (2011)); TgiCL Assembler (Pertea, et al., Bioinformatics 19(5):651-52 (2003)): Maq (Mapping and Assembly with Qualities) by Heng Li, available for download through the SourceForge website maintained by Geeknet (Fairfax, Va.); MIRA3 (Mimicking Intelligent Read Assembly), described in Chevreux, B., et al., Genome Sequence Assembly Using Trace Signals and Additional Sequence Information, 1999, Computer Science and Biology: Proceedings of the German Conference on Bioinformatics (GCB) 99:45-56; PGA4genomics (described in Zhao F., et al., Genomics, 94(4):284-6 (2009)); and Phrap (described, e.g., in de la Bastide, M. and McCombie, W. R., Current Protocols in Bioinformatics, 17:11.4.1-11.4.15 (2007)). CLC cell is a de Bruijn graph-based computer program for read mapping and de novo assembly of NGS reads available from CLC bio Germany (Muehltal, Germany).

The invention provides for positioning a consensus sequence or a contig along a reference genome. In certain embodiments, a consensus sequence or a contig is positioned on a reference through information from known molecular markers or probes. In some embodiments, protein-coding sequence data in a contig, consensus sequence, or reference genome is represented by amino acid sequence and a contig is positioned along a reference genome. In some embodiments, a consensus sequence or a contig is positioned by an alignment of the contig to a reference genome.

Alignment, as used herein, generally involves placing one sequence along another sequence, iteratively introducing gaps along each sequence, scoring how well the two sequences match, and preferably repeating for various positions along the reference. The best-scoring match is deemed to be the alignment and represents an inference about the historical relationship between the sequences. Mapping or aligning the sequence reads can also be used to identify variant sequences. These variant sequences may encompass genetic mutations, such as substitutions, insertions, or deletions. For example, a base in the read alongside a non-matching base in the reference indicates that a substitution mutation has occurred at that point. Similarly, where one sequence includes a gap alongside a base in the other sequence, an insertion or deletion mutation (an “indel”) is inferred to have occurred. When it is desired to specify that one sequence is being aligned to one other, the alignment is sometimes called a pairwise alignment. Multiple sequence alignment generally refers to the alignment of two or more sequences, including, for example, by a series of pairwise alignments.

In some embodiments, scoring an alignment involves setting values for the probabilities of substitutions and indels. When individual bases are aligned, a match or mismatch contributes to the alignment score by a substitution probability, which could be, for example, 1 for a match and 0.33 for a mismatch. An indel deducts from an alignment score by a gap penalty, which could be, for example, −1. Gap penalties and substitution probabilities can be based on empirical knowledge or a priori assumptions about how sequences mutate. Their values affect the resulting alignment. Particularly, the relationship between the gap penalties and substitution probabilities influences whether substitutions or indels will be favored in the resulting alignment.

Stated formally, an alignment represents an inferred relationship between two sequences, x and y. For example, in some embodiments, an alignment A of sequences x and y maps x and y respectively to another two strings x′ and y′ that may contain spaces such that: (i) |x′|=|y′|; (ii) removing spaces from x′ and y′ should get back x and y, respectively; and (iii) for any i, x′[i] and y′[i] cannot be both spaces.

A gap is a maximal substring of contiguous spaces in either x′ or y′. An alignment A can include the following three kinds of regions: (i) matched pair (e.g., x′[i]=y′[i]; (ii) mismatched pair, (e.g., x′[i]≠y′[i] and both are not spaces); or (iii) gap (e.g., either x′[i . . . j] or y′[i . . . j] is a gap). In certain embodiments, only a matched pair has a high positive score a. In some embodiments, a mismatched pair generally has a negative score b and a gap of length r also has a negative score g+rs where g, s<0. For DNA, one common scoring scheme (e.g. used by BLAST) makes score a=1, score b=−3, g=−5 and s=−2. The score of the alignment A is the sum of the scores for all matched pairs, mismatched pairs and gaps. The alignment score of x and y can be defined as the maximum score among all possible alignments of x and y.

In some embodiments, any pair has a score a defined by a 4×4 matrix B of substitution probabilities. For example, B(i,i)=1 and 0<B(i,j)_{i< >j}<1 is one possible scoring system. For instance, where a transition is thought to be more biologically probable than a transversion, matrix B could include B(C,T)=0.7 and B(A,T)=0.3, or any other set of values desired or determined by methods known in the art.

Alignment according to some embodiments of the invention includes pairwise alignment. A pairwise alignment, generally, involves—for sequence Q (query) having m characters and a reference genome T (target) of n characters-finding and evaluating possible local alignments between Q and T. For any 1≤i≤n and 1≤j≤m, the largest possible alignment score of T[h . . . i] and Q[k . . . j], where h≤i and k≤j, is computed (i.e. the best alignment score of any substring of T ending at position i and any substring of Q ending at position j). This can include examining all substrings with cm characters, where c is a constant depending on a similarity model, and aligning each substring separately with Q. Each alignment is scored, and the alignment with the preferred score is accepted as the alignment. In some embodiments an exhaustive pairwise alignment is performed, which generally includes a pairwise alignment as described above, in which all possible local alignments (optionally subject to some limiting criteria) between Q and T are scored.

In some embodiments, pairwise alignment proceeds according to dot-matrix methods, dynamic programming methods, or word methods. Dynamic programming methods generally implement the Smith-Waterman (SW) algorithm or the Needleman-Wunsch (NW) algorithm. Alignment according to the NW algorithm generally scores aligned characters according to a similarity matrix S(a,b) (e.g., such as the aforementioned matrix B) with a linear gap penalty d. Matrix S(a,b) generally supplies substitution probabilities. The SW algorithm is similar to the NW algorithm, but any negative scoring matrix cells are set to zero. The SW and NW algorithms, and implementations thereof, are described in more detail in U.S. Pat. No. 5,701,256 and U.S. Pub. 2009/0119313, both herein incorporated by reference in their entirety. Computer programs known in the art for implementing these methods are described in more detail below.

In certain embodiments, an exhaustive pairwise alignment is avoided by positioning a consensus sequence or a contig along a reference genome through the use of a transformation of the sequence data. One useful category of transformation according to some embodiments of the invention involves making compressed indexes of sequences (see, e.g., Lam, et al., Compressed indexing and local alignment of DNA, 2008, Bioinformatics 24(6):791-97). Exemplary compressed indexes include the FN-index, the compressed suffix array, and the Burrows-Wheeler Transform (BWT, described in more detail below).

An alignment according to the invention can be performed using any suitable computer program known in the art.

One exemplary alignment program, which implements a BWT approach, is Burrows-Wheeler Aligner (BWA) available from the SourceForge web site maintained by Geeknet (Fairfax, Va.). BWA can align reads, contigs, or consensus sequences to a reference. BWT occupies 2 bits of memory per nucleotide, making it possible to index nucleotide sequences as long as 4G base pairs with a typical desktop or laptop computer. The pre-processing includes the construction of BWT (i.e., indexing the reference) and the supporting auxiliary data structures.

BWA implements two different algorithms, both based on BWT. Alignment by BWA can proceed using the algorithm bwa-short, designed for short queries up to ˜200 bp with low error rate (<3%) (Li H. and Durbin R. Bioinformatics, 25:1754-60 (2009)). The second algorithm, BWA-SW, is designed for long reads with more errors (Li H. and Durbin R. (2010) Fast and accurate long-read alignment with Burrows-Wheeler Transform. Bioinformatics, Epub.). The BWA-SW component performs heuristic Smith-Waterman-like alignment to find high-scoring local hits. One skilled in the art will recognize that bwa-sw is sometimes referred to as “bwa-long”, “bwa long algorithm”, or similar. Such usage generally refers to BWA-SW. An alignment program that implements a version of the Smith-Waterman algorithm is MUMmer, available from the SourceForge web site maintained by Geeknet (Fairfax, Va.). MUMmer is a system for rapidly aligning entire genomes, whether in complete or draft form (Kurtz, S., et al., Genome Biology, 5:R12 (2004); Delcher, A. L., et al., Nucl. Acids Res., 27:11 (1999)). For example, MUMmer 3.0 can find all 20-basepair or longer exact matches between a pair of 5-megabase genomes in 13.7 seconds, using 78 MB of memory, on a 2.4 GHz Linux desktop computer. MUMmer can also align incomplete genomes; it can easily handle the 100s or 1000s of contigs from a shotgun sequencing project, and will align them to another set of contigs or a genome using the NUCmer program included with the system. If the species are too divergent for a DNA sequence alignment to detect similarity, then the PROmer program can generate alignments based upon the six-frame translations of both input sequences.

Another exemplary alignment program according to embodiments of the invention is BLAT from Kent Informatics (Santa Cruz, Calif.) (Kent, W. J., Genome Research 4: 656-664 (2002)). BLAT (which is not BLAST) keeps an index of the reference genome in memory such as RAM. The index includes of all non-overlapping k-mers (except optionally for those heavily involved in repeats), where k=11 by default. The genome itself is not kept in memory. The index is used to find areas of probable homology, which are then loaded into memory for a detailed alignment.

Another alignment program is SOAP2, from Beijing Genomics Institute (Beijing, Conn.) or BGI Americas Corporation (Cambridge, Mass.). SOAP2 implements a 2-way BWT (Li et al., Bioinformatics 25(15):1966-67 (2009); Li, et al., Bioinformatics 24(5):713-14 (2008)).

Another program for aligning sequences is Bowtie (Langmead, et al., Genome Biology, 10:R25 (2009)). Bowtie indexes reference genomes by making a BWT.

Other exemplary alignment programs include: Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator from Real Time Genomics, Inc. (San Francisco, Calif.); Novoalign from Novocraft (Selangor, Malaysia); Exonerate, European Bioinformatics Institute (Hinxton, UK) (Slater. G., and Birney. E., BMC Bioinformatics 6:31 (2005)), Clustal Omega, from University College Dublin (Dublin, Ireland) (Sievers F., et al., Mol Syst Biol 7, article 539 (2011)); ClustalW or ClustalX from University College Dublin (Dublin, Ireland) (Larkin M. A., et al., Bioinformatics, 23, 2947-2948 (2007)); and FASTA. European Bioinformatics Institute (Hinxton, UK) (Pearson W. R., et al., PNAS 85(8):2444-8 (1988); Lipman, D. J., Science 227(4693):1435-41 (1985)).

One exemplary method for matching sequence reads is provided in Krawitz et al., Bioinformatics, Vol 26. No. 6 (2010), incorporated herein by reference in its entirety. Short reads were mapped to the reference genome using BWA 0.4.9 (Li and Durbin, 2010), Novoalign Release 2.05.02 (Hercus, 2009) and RazerS1.0 (Weese et al., 2009) with default settings for mismatch penalty, gap opening penalty, and gap extension penalty. For variant detection, Bowtie 0.11.3 (Langmead et al., 2009) and MAQ 0.7.1 (Li et al., 2008) were also tested with their default settings, which do not allow gapped alignments. MAQ uses a spaced-seed approach to align reads. With default settings, only reads that map to the reference genome with less than three mismatched bases in the first 28 bases of the read were aligned. The ungapped alignments with the best alignment score were reported. Bowtie and BWA are based on backward search schemes with a Burrows-Wheeler transformation to efficiently align short sequencing reads against large reference sequences. Bowtie allows two mismatches or fewer within the high-quality end of each read, and it places an upper limit on the sum of the quality values at mismatched alignment positions. Novoalign finds global optimum alignments using full Needleman-Wunsch algorithm with affine gap penalties. RazerS adapts a q-gram counting technique for read filtering and maps reads using edit or Hamming distance as thresholds. All alignments were converted to Sequence Alignment/Map (sam) format that codes the position of an indel in the short-read in CIGAR string format. The consensus sequence was called according to the MAQ consensus model (U et al., 2008) with samtools release 0.1.7.

Other methods can be used in accordance with the invention to match sequence reads. In one embodiment, the sequence reads are assembled into contigs which are aligned with the reference sequence. With each contig aligned to the reference genome, any differences between the contig and the reference genome can be identified by a comparison. For example, a computer alignment program can report any non-matching pair of aligned bases, e.g., in a visual display or as a character string. Since the entire contig is aligned to the reference genome, instead of individual short reads, an incorrect position is improbable. Thus, any indels introduced typically will represent differences between the nucleic acid and the reference.

The position of an indel with respect to the reference sequence is not necessarily unequivocally defined by a single coordinate. Accordingly, methods encompassed by the invention include determining the equivalent insertion/deletion region (EIR) of the variant sequence. For example, the insertion of an adenosine into a sequence motif of CᵢAᵢ₊₁Aᵢ₊₂Gᵢ₊₃ after position i results in a variant sequence that is identical to the sequence produced by an inserted adenosine after positions i+1 or i+2. Therefore, to unambiguously annotate this insertion, listing all equivalent indel positions, i.e., +A{i, i+1, i+2} is required. This is so because the position of the inserted adenosine is not unambiguously defined by a single coordinate, but only by the set of equivalent positions. In certain embodiments, when a read is aligned with a gap, the equivalent indel region (EIR) is determined by computing all equivalent positions with respect to the sequence of this specific insertion or deletion. Exemplary algorithms and further details on determining EIR are provided in Krawitz et al., Bioinformatics, Vol. 26, No. 6 (2010), incorporated by reference herein in its entirety. For example, suitable algorithms identify all called indel positions that lead to the identical mutated sequence. One such algorithm for determination of EIR is as follows and described in detail in Krawitz et al. It is understood that this exemplary algorithm is not limiting and that other algorithms could be used in accordance with the present invention.

| Input: sequence s, position iₚ, pattern p | | |
|---|---|---|
| Output: EIR | | |
| 1. | x ← p; | // Extend eir to the right |
| 2. | ir ← iₚ; | |
| 3. | r ← s_{ir + 1}; | |
| 4. | x’ ← x₂...xₖ.x₁; | |
| 5. | while (x.r ==r.x’) do (6-9) | |
| 6. | x ← x’; | |
| 7. | ir ← iᵣ + 1; | |
| 8. | r ← sᵢᵣ + 1; | |
| 9. | x’ ← x₂...xₖ.x₁; | |
| 10. | x ←p; | // Extend eir to the left |

| | |
|---|---|
| 11. | il ← iₚ; |
| 12. | l ←sᵢₗ₋₁; |
| 13. | x’ ← xₖ.x₁...xₖ₋₁; |
| 14. | while (l.x==x’.1) do (15-18) |
| 15. | x ←x’; |
| 16. | iₗ ←iₗ−1; |
| 17. | l ← sᵢₗ₋₁; |
| 18. | x’ ←xₖ.x₁...xₖ₋₁; |
| 19. | eir ← {x, il, ir}; |
| 20. | return eir |

In this exemplary algorithm, an EIR is computed from the genomic sequence s around an indel of a sequence pattern p after position iₚ. iᵣ denotes the rightmost position of the EIR and r the nucleotide to the right of iᵣ. Line 4 calculates acyclic permutation x′ of the pattern in x. The ‘.’ operator indicates a string concatenation. Lines 1-9 extend the EIR to the right. Following the extension to the left (lines 10-18), the left and rightmost positions are returned together with the leftmost pattern.

The usefulness of determining the EIR is particular appropriate in the case of tandem repeats. Tandem repeats comprise short DNA sequences or “units,” where the unit is repeated several times and the repetitions are adjacent to one another. Mutations within the tandem repeat are associated with various diseases. Huntington disease, for example, is associated with the expansion of the number of tandem repeats located within a specific disease. Tandem repeats are relatively unstable, with mutation rates typically 10 to 100,000 times higher than in other parts of the genome. Most mutations in tandem repeats are not due to point mutations but rather to repeat polymorphisms that occur when the number of the repeating unit changes. In other words, the mutations are attributable to the insertion or deletions of units within the tandem repeat. In the case of tandem repeats, it is especially difficult to define an indel by a single position. Accordingly, certain embodiments of the invention include determining the EIR of a variant sequence associated with a tandem repeat. In these cases, the EIR of the variant is essentially the contiguous block of DNA representing its associated tandem repeat.

There are currently two major models that describe the mechanisms by which tandem repeats expand or contract: strand-slippage replication and recombination. Strand-slippage replication occurs during replication of the tandem repeat DNA when there is mispairing between the template and nascent DNA strands. When the newly synthesized strand denatures from the template strand during synthesis of the tandem repeat sequence, it will occasionally pair with another part of the repeat sequence. If the template strand is looped out, then contraction (i.e., deletion) of the tandem repeat occurs. If the nascent strand loops out, then an expansion (i.e., insertion) will result. Recombination events, including unequal crossing over and gene conversion, can also lead to contraction and expansions of tandem repeat sequences. Further detail on these processes is provided in Gemayel et al, Annu. Rev. Genet, 2010. 44:445-77, herein incorporated by reference in its entirety.

Due to the biological mechanisms mentioned above, naturally occurring insertion and deletion mutations tend to occur at tandem repeats (i.e., within EIRs) much more frequently than would be expected by chance. This phenomenon can be exploited to distinguish true variants or mutations from false positives (i.e., variants arising from sequencing artifacts). For example, true mutations are often associated with EIRs greater than one base pair in length because the insertion or deletion lesion can be shifted on the genome due to its sequence and the adjacent sequence context. See, for example, the earlier sequence motif of CᵢAᵢ₊₁Aᵢ₊₂Gᵢ₊₃. In contrast, an EIR may be one base pair if (i) the corresponding variant is a non-insertion or non-deletion (i.e., a substitution) or (ii) the insertion or deletion is outside of a tandem repeat. In these situations, the EIR length of one base pair is more likely to correspond to a sequencing artifact rather than a true mutation.

In addition, appropriate probability-based scores can be used to measure the mutual dependence between insertions and deletions to further reduce uncertainty regarding whether an identified variant represents a true positive (e.g., a true mutation) or a false positive. For example:

p(deletion|repeat)=p(repeat|deletion)p(deletion)/p(repeat)

where p(repeat|deletion) is the likelihood of a repeat given the deletion, p(deletion) is the prior probability of a deletion in the absence of additional evidence, a p(repeat) is a normalization factor that accounts for local variability in sequence repetitiveness. The latter two values depend on the specific genomic regions under consideration. This means that it is likely that probabilities would be calculated separately for different sized variants. In combination with other pieces of evidence, such as genotype qualities, a simple reference table would provide additional confidence in any particular variant call given its presence in a repetitive region. This technique assumes that erroneously placed insertions and deletions (i.e., false positives) resulting from alignment artifacts will not have bias with respect to the sequence context.

The invention further contemplates identifying a functional region that comprises at least a portion of the EIR. Consistent annotation of variant sequences requires implementing rules or performing simulations that consider variant sequences in both the genomic and functional context. The functional context of the variant sequence is the protein coding space associated with the variant sequence. In some embodiments, functional annotation regions are described using known gene structures. These gene structures may include, but are not limited to, genes, exons, introns, splice sites, codons, regulatory elements, and non-coding regions. It is contemplated by the invention that the EIR of a variant sequence may actually span multiple annotations with different clinical significances. In certain embodiments, functional annotation of the variant sequence is performed by an algorithm that applies the appropriate rules or simulations. One exemplary algorithm is provided below.

The invention also involves associating the genomic coordinates of a variant sequence (provided by the EIR) to their local functional (protein coding region) coordinates. The scope of the functional annotation will change depending on the variant in question. Substitutions, for example, are relatively uncomplicated because they will map to a single codon or splice site. Indels associated with tandem repeats, however, are relatively more complex due to the possibility that they may span more than one functional region.

In some embodiments of the invention, annotation is performed using the smallest possible functional region or set of regions as the case may be. For example, all substitutions can be annotated at the level of a single codon. Similarly, an EIR-associated indel can be annotated with its spanning codons/splice sites rather than the entire coding region.

The invention also involves determining the clinical significance of the variant sequence by associating its EIR with the identified functional region. In certain embodiments, associating the EIR with the functional region means attempting to push the variant EIR completely out of the functional region by retrieving the extreme lower or upper position of the variant EIR. Selecting the upper or lower position depends on the orientation of the variant relative to its associated functional region or regions. If the variant can be pushed completely out of its functional region, the variant sequence is not clinically significant.

In other words, determining the clinical significance of the variant sequence involves asking whether the variant EIR extends beyond the functional region. If it does, the variant can be “pushed” out of the functional region, i.e., its position is ambiguous and therefore, has no functional effect because it could lie outside the functional region.

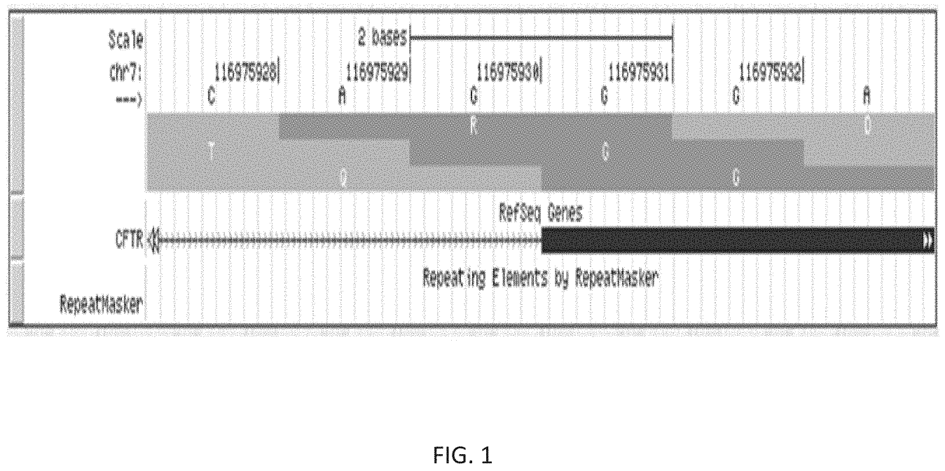

This concept is demonstrated in the following example. A simple base pair homopolymeric repeat (GGG) is provided in FIG. 1, which partially overlaps the exon boundary and its associated splice site (chr7: 116975929-116975930). Depending on its size, a deletion of one or more nucleotides from this repeat may be reported by detection algorithms at any of three equivalent positions (chr7: 116975929-116975931) within the EIR chr7: 116975929-chr7: 116975932; however, in this particular case, the functional annotation depends on the exact position of the variant. Translating genomic positions into their functional analogues would lead to a SPLICESITE annotation for chr7: 116975929deG, whereas the equivalent chr7:116975931delG is a FRAMESHIFT. In the provided example, algorithms contemplated by the invention may classify any of the three equivalent single base pair deletions as a FRAMESHIFT. In the provided example, variants of this type do not disturb the splice site sequence (chr7:116975929-116975930), and therefore, are not clinically significant in this case. Likewise, chr7:116975929delGG, chr7:116975929insGG and their equivalents are also FRAMESHIFT, whereas chr7:116975929delGGG is SPLICESITE (clinically significant in this case) and chr7:116975929insGGG is inframe.

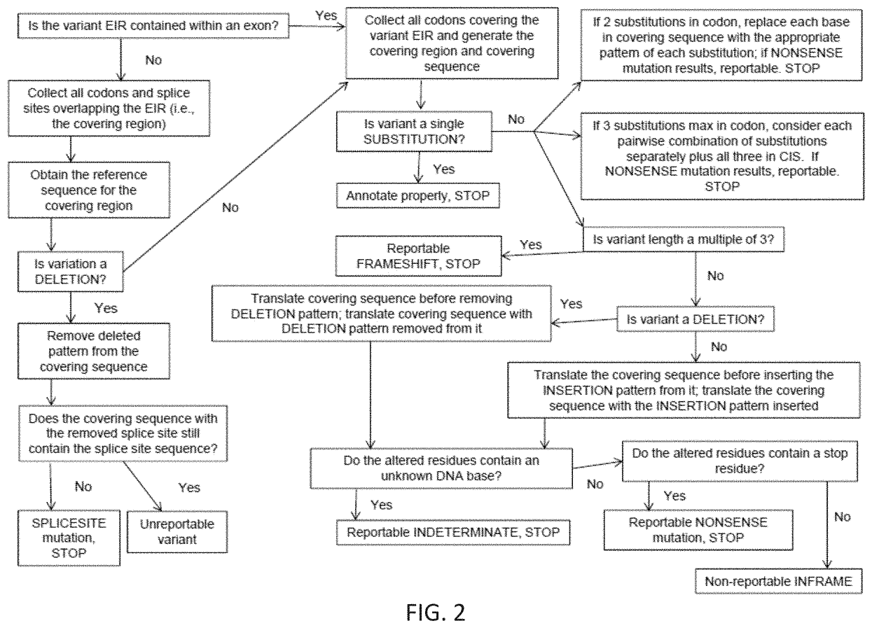

The determination of clinical significance by associating the EIR of the variant sequence to the functional region is also demonstrated through the algorithm provided below. In the following algorithm, the term reportable means that the variant is clinically significant, while the term non-reportable or unreportable means that that the variant is not clinically significant, i.e benign or unknown. In the provided algorithm, clinical significance, or the lack thereof, is associated with mutations of a certain type, including but not limited to, splice site mutations, in-frame mutations, nonsense mutations, frameshift mutations, and mutations comprising an unknown nucleic acid base. These mutations may be clinically significant in the functional context as they may affect the functionality or expression of the associated protein. It is understood that the algorithm provided below is not limiting and that other algorithms may be used in accordance with the present invention. In addition, while it is contemplated that certain embodiments of the invention use algorithms to associate EIRs to functional regions, the invention is not limited to such uses. Associations can be made, for example, by consulting a reference table. A flow chart outlining the steps of the algorithm is provided in FIG. 2.

Step 1: Determine if the variant EIR is contained within an exon, if yes go to step 3, if no go to step 2a.

Step 2a: Collect all codons and splice sites overlapping the EIR (also known as the covering region)

Step 2b: Get the reference sequence for the covering region (also known as the covering sequence)

Step 2c: If the variant is a DELETION remove the deleted pattern from the covering sequence and go to step 2d, otherwise go to step 3

Step 2d: If the covering sequence with the deleted pattern removed still contains the splice site sequence, this is an unreportable variant (i.e., the allele will still be properly spliced), otherwise this is a SPLICESITE mutation, STOP

Step 3: Collect all codons covering the variant EIR and generate the covering region and covering sequence as described in Steps 2a and 2b

Step 4: If the variant is a single SUBSTITUTION, annotate appropriately, STOP (singleton substitutions that map to a single codon or splice site are relatively uncomplicated), otherwise if the covering codons include more than one SUBSTITUTION go to step 4a.

Substitutions that are adjacent in the gene product (protein) may be separated by arbitrary distances within a genomic context due to intervening (intronic) sequence. Therefore, special consideration is taken to ensure that covering sequence is accurate for both internal codons (completely contained within an exon) as well as codon fragments (across exon boundaries).

Step 4a: Multiple substitutions within the same codon may be present within the same contiguous stretch of DNA (CIS) or on different contiguous stretches (TRANS) with potentially different clinical effects. Therefore, certain embodiments contemplate exploring cis-trans associations either directly using individual read data or indirectly by exploring all possible combinations, which is small and therefore computationally tractable. Trans associations are considered independently as described in step 4. If there are two (2) substitutions in the codon go to step 4b, else there are a maximum of three (3) substitutions in the codon, go to step 4c

Step 4b: Replace each base (letter) in the covering sequence with the appropriate pattern of each substitution. If a NONSENSE codon results the variant is reportable, STOP

Step 4c: Consider each (of three) pairwise combination of substitutions separately (according to Step 4b) as well as all three in CIS. If a NONSENSE codon results the variant is reportable, STOP

Step 5: If the variant length is not a multiple of 3 (codon length), this is a reportable FRAMESHIFT, STOP

Step 6: If the variant is a DELETION go to step 7a, otherwise go to step 8a

Step 7a: Translate the covering sequence before removing the DELETION pattern from it

Step 7b: Translate the covering sequence with the DELETION pattern removed from it (i.e., the set of altered residues), got o step 9

Step 8a: Translate the covering sequence before inserting the INSERTION pattern from it

Step 8b: Translate the covering sequence with the INSERTION pattern inserted (i.e., the altered residues), go to step 9

Step 9: If the altered residues contain an unknown DNA base (e.g., “N”) the allele is reportable INDETERMINATE, STOP. Otherwise, go to step 10

Step 10: If the altered residues contain a stop residue (‘*’) the variant is reportable NONSENSE, STOP. Otherwise go to step 11

Step 11: The variant is a non-reportable INFRAME

In certain embodiments, the process of applying EIR-assisted confidence scores and functional annotations to determine clinical significance can be reduced to the following steps. In the first step, one would determine if an identified variant is associated with a particular disease, for example, by consulting a relevant database. If the variant is not known to be disease-causing, then it is deemed novel. If the variant is a substitution, its clinical impact can be determined directly from its genomic coordinate for reasons discussed above. If the reference is not a substitution, the EIR of the variant is determined using the described methods. If the variant EIR length is equal to one base pair, the probability that the variant is a false positive is assessed, for reasons discussed above. If it is determined that variant is real, i.e., a true mutation, the next step is to annotate the variant EIR with all the appropriate functional information. As discussed, these functional annotation regions may include, without limitation, genes, exons, introns, splice sites, codons, non-coding regions, etc. Next, the EIR of the variant sequence is associated to the annotated functional region. In some embodiments, this involves determining whether the EIR extends beyond the functional region. If it does, the variant can be “pushed” out of the functional region and thus, has no functional effect. If the variant can be pushed completely out of the functional region, the result is unreported (or reported as benign or unknown), otherwise the clinical significance of the variant is determined and reported accordingly.

In some embodiments, any or all of the steps of the invention are automated. For example, a Perl script or shell script can be written to invoke any of the various programs discussed above (see, e.g., Tisdall, Mastering Perl for Bioinformatics, O'Reilly & Associates, Inc., Sebastopol, C A 2003; Michael, R., Mastering Unix Shell Scripting, Wiley Publishing, Inc., Indianapolis, Ind. 2003). Alternatively, methods of the invention may be embodied wholly or partially in one or more dedicated programs, for example, each optionally written in a compiled language such as C++ then compiled and distributed as a binary. Methods of the invention may be implemented wholly or in part as modules within, or by invoking functionality within, existing sequence analysis platforms. In certain embodiments, methods of the invention include a number of steps that are all invoked automatically responsive to a single starting queue (e.g., one or a combination of triggering events sourced from human activity, another computer program, or a machine). Thus, the invention provides methods in which any or the steps or any combination of the steps can occur automatically responsive to a queue. Automatically generally means without intervening human input, influence, or interaction (i.e., responsive only to original or pre-queue human activity).

The invention also encompasses various forms of output, which includes an accurate and sensitive interpretation of the subject nucleic acid. The output can be provided in the format of a computer file. In certain embodiments, the output is a FASTA file, VCF file, text file, or an XML file containing sequence data such as a sequence of the nucleic acid aligned to a sequence of the reference genome. In other embodiments, the output contains coordinates or a string describing one or more mutations in the subject nucleic acid relative to the reference genome. Alignment strings known in the art include Simple UnGapped Alignment Report (SUGAR), Verbose Useful Labeled Gapped Alignment Report (VULGAR), and Compact Idiosyncratic Gapped Alignment Report (CIGAR) (Ning, Z., et al., Genome Research 11(10):1725-9 (2001)). These strings are implemented, for example, in the Exonerate sequence alignment software from the European Bioinformatics Institute (Hinxton, UK).

In some embodiments, the output is a sequence alignment-such as, for example, a sequence alignment map (SAM) or binary alignment map (BAM) file-comprising a CIGAR string (the SAM format is described, e.g., in Li, et al., The Sequence Alignment/Map format and SAMtools, Bioinformatics, 2009, 25(16):2078-9). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The cigar string defines the sequence of matches/mismatches and deletions (or gaps). For example, the cigar string 2MD3M2D2M will mean that the alignment contains 2 matches, 1 deletion (number 1 is omitted in order to save some space), 3 matches, 2 deletions and 2 matches.

As contemplated by the invention, the functions described above can be implemented using software, hardware, firmware, hardwiring, or any combinations of these. Features implementing functions can also be physically located at various positions, including being distributed such that portions of functions are implemented at different physical locations.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

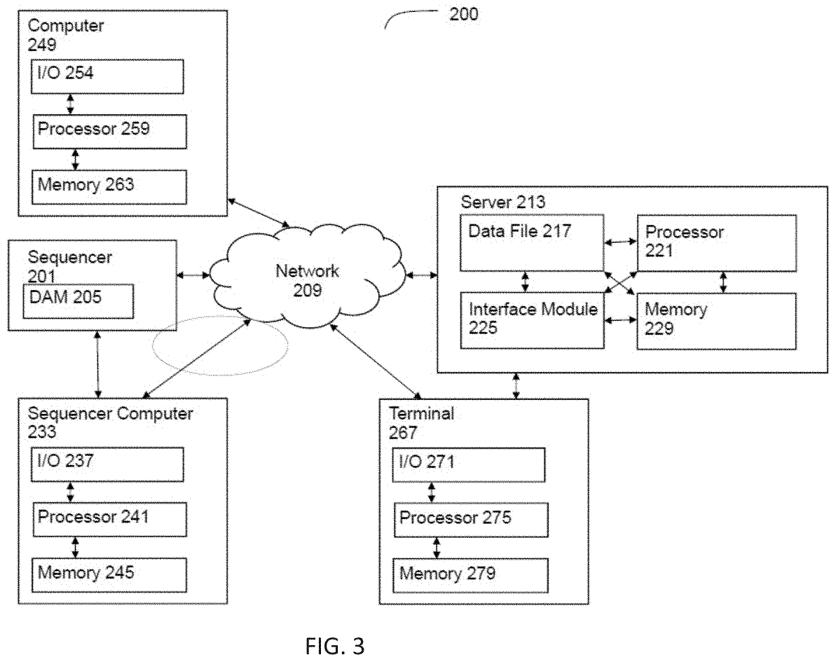

In an exemplary embodiment shown in FIG. 3, system 200 can include a sequencer 201 with data acquisition module 205 to obtain sequence read data. Sequencer 201 may optionally include or be operably coupled to its own, e.g., dedicated, sequencer computer 233 (including an input/output mechanism 237, one or more of processor 241 and memory 245). Additionally or alternatively, sequencer 201 may be operably coupled to a server 213 or computer 249 (e.g., laptop, desktop, or tablet) via network 209. Computer 249 includes one or more processor 259 and memory 263 as well as an input/output mechanism 254. Where methods of the invention employ a client/server architecture, an steps of methods of the invention may be performed using server 213, which includes one or more of processor 221 and memory 229, capable of obtaining data, instructions, etc., or providing results via interface module 225 or providing results as a file 217. Server 213 may be engaged over network 209 through computer 249 or terminal 267, or server 213 may be directly connected to terminal 267, including one or more processor 275 and memory 279, as well as input/output mechanism 271.

System 200 or machines according to the invention may further include, for any of I/O 249, 237, or 271 a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory 263, 245, 279, or 229 according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, solid-state memories (e.g., subscriber identity module (SIM) card, secure digital card (SD card), micro SD card, or solid-state drive (SSD)), optical and magnetic media, and any other tangible storage media.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1-10. (canceled)

11. A method for determining a clinically significant variant sequence within a sequence read of a biological sample obtained from an organism, the method comprising:
sequencing, by a sequencer, nucleic acid to generate a plurality of sequence reads including the sequence read of the biological sample, wherein the nucleic acid was extracted from the biological sample, and at least the sequence read of the biological sample includes a sequence of nucleotide data less than about 150 bases in length;
receiving, by a computer included in or coupled to the sequencer, a reference sequence;
aligning, by the computer, at least the sequence read of the biological sample with the reference sequence;
identifying, by the computer, a variant sequence within the sequence read of the biological sample based on the aligning the sequence read with the reference sequence;
determining, by the computer and based on information from a variant database, that the identified variant sequence is a novel variant sequence not known to be disease-causing, wherein the variant database stores variant sequences associated with diseases;
generating, by the computer, an equivalent insertion or deletion region (EIR) of the variant sequence, the EIR comprising a region of nucleic acid representing equivalent positions of the variant sequence with respect to the reference sequence;
identifying, by the computer, a functional region that includes at least a portion of the EIR, wherein the functional region is described by gene structures and obtained by annotating the EIR of the variant sequence with protein coding region coordinates;
determining, by the computer, whether or not the EIR extends beyond the functional region; and
determining, by the computer, either that the variant sequence has clinical significance when the EIR does not extend beyond the functional region, or that the variant sequence does not have clinical significance when the EIR extends beyond the functional region.

12. The method of claim 11, wherein the variant sequence is determined to have clinical significance.

13. The method of claim 11, wherein the annotating the EIR of the variant sequence includes associating genomic coordinates of the variant sequence to the protein coding region coordinates.

14. The method of claim 11, wherein the gene structures include genes, exons, introns, splice sites, codons, or non-coding regions.

15. The method of claim 11, wherein the variant sequence is located in a tandem repeat.

16. The method of claim 11, wherein the variant sequence includes a splice site mutation, an in-frame mutation, a nonsense mutation, or a frameshift mutation.

17. The method of claim 11, wherein the biological sample includes haploid or diploid cells.

18. A computer system, comprising:
a sequencer configured to sequence nucleic acid to generate a plurality of sequence reads, wherein the nucleic acid was extracted from a biological sample obtained from an organism, and at least a sequence read of the biological sample includes a sequence of nucleotide data less than about 150 bases in length;
a processor coupled to the sequencer, and configured to:
receive a reference sequence;
align at least the sequence read of the biological sample with the reference sequence;
identify a variant sequence within the sequence read based on the aligned sequence read with the reference sequence;
determine, based on information from a variant database, the identified variant sequence is a novel variant sequence that is not known to be disease-causing, wherein the variant database stores variant sequences associated with diseases;
generate an equivalent insertion or deletion region (EIR) of the variant sequence, the EIR comprising a region of nucleic acid representing equivalent positions of the variant sequence with respect to the reference sequence;
identify a functional region that includes at least a portion of the EIR, wherein the functional region is described by gene structures and obtained by annotating the EIR of the variant sequence with protein coding region coordinates;
determine whether or not the EIR extends beyond the functional region; and
determine, either that the variant sequence has clinical significance when the EIR does not extend beyond the functional region, or that the variant sequence does not have clinical significance when the EIR extends beyond the functional region.

19. The computer system of claim 18, wherein the variant sequence is determined to have clinical significance.

20. The computer system of claim 18, wherein the annotating the EIR of the variant sequence includes associating genomic coordinates of the variant sequence to the protein coding region coordinates.

21. The computer system of claim 18, wherein the gene structures include genes, exons, introns, splice sites, codons, or non-coding regions.

22. The computer system of claim 18, wherein the variant sequence is located in a tandem repeat.

23. The computer system of claim 18, wherein the variant sequence includes a splice site mutation, an in-frame mutation, a nonsense mutation, or a frameshift mutation.

24. The computer system of claim 18, wherein the biological sample includes haploid or diploid cells.

25. A non-transitory computer-readable medium having instructions stored thereon that, when executed by at least one computing device, cause the at least one computing device to perform operations comprising:
receiving a plurality of sequence reads including a sequence read sequenced from a nucleic acid, wherein the nucleic acid was extracted from a biological sample obtained from an organism, and the sequence read includes a sequence of nucleotide data less than about 150 bases in length;
receiving a reference sequence;
aligning the sequence read with the reference sequence;
identifying a variant sequence within the sequence read based on the aligning the sequence read with the reference sequence;
determining, based on information from a variant database, whether the identified variant sequence is a novel variant sequence that is not known to be disease-causing, wherein the variant database includes variant sequences associated with diseases;
generating an equivalent insertion or deletion region (EIR) of the variant sequence, the EIR comprising a region of nucleic acid representing equivalent positions of the variant sequence with respect to the reference sequence;
identifying a functional region that includes at least a portion of the EIR, wherein the functional region is described by gene structures and obtained by annotating the EIR of the variant sequence with protein coding region coordinates;
determining whether or not the EIR extends beyond the functional region; and
determining either that the variant sequence has clinical significance when the EIR does not extend beyond the functional region, or that the variant sequence does not have clinical significance when the EIR extends beyond the functional region.

26. The non-transitory computer-readable medium of claim 25, wherein when the variant sequence is determined to have clinical significance.

27. The non-transitory computer-readable medium of claim 25, wherein the annotating the EIR of the variant sequence includes associating genomic coordinates of the variant sequence to the protein coding region coordinates.

28. The non-transitory computer-readable medium of claim 25, wherein the gene structures include genes, exons, introns, splice sites, codons, or non-coding regions.

29. The non-transitory computer-readable medium of claim 25, wherein the variant sequence is located in a tandem repeat.

30. The non-transitory computer-readable medium of claim 25, wherein the variant sequence includes a splice site mutation, an in-frame mutation, a nonsense mutation, or a frameshift mutation.
